(19) United States
(12) Patent Application Publication
(10) Pub. No.: US 2024/0156907 A1
(43) Pub. Date: May 16, 2024

(54) NOVEL FORMULATION OF FUSION PROTEIN

(71) Applicant: Kashiv BioSciences, LLC, Piscataway, NJ (US)

(72) Inventors: Om Narayan, Ahmedabad (IN); Tarun Kumar Gupta, Ahmedabad (IN); Kiran Suresh Avadhani, Ahmedabad (IN); Kaushal Joshi, Ahmedabad (IN)

(21) Appl. No.: 18/550,920

(22) PCT Filed: Mar. 16, 2022
(86) PCT No.: PCT/IB2022/052377

§ 371 Date: Sep. 15, 2023

(30) Foreign Application Priority Data
Mar. 16, 2021 (IN) 202121011096

(51) Int. Cl. A61K 38/17; A61K 47/10; A61K 47/22

(52) U.S. Cl. CPC A61K 38/179; A61K 38/177; A61K 38/1774; A61K 47/10; A61K 47/22

(57) ABSTRACT

The present invention relates to novel liquid formulations comprising pharmacologically active fusion protein. The present invention discloses the use of histidine buffer in combination with other excipients to stabilize the fusion protein by lowering the product related impurities. In another aspects invention provides a formulation of fusion protein with low viscosity.

### FIELD OF THE INVENTION

The present invention relates to novel liquid formulations comprising pharmacologically active fusion protein. The present invention discloses the use of histidine buffer in combination with other excipients to stabilize the fusion protein by lowering the product related impurities. In another aspects invention provides a formulation of fusion protein with low viscosity.

### BACKGROUND OF THE INVENTION

In earlier days antibody formulations were stabilised with lyophilisation of protein and were reconstituted prior to use with appropriate solvent system. However, with time requirement of high concentrated and stable liquid formulation increased. As fusion proteins are not very stable, high concentration of fusion protein in liquid formulation often results in precipitation of fusion protein and degradation of the same. Further it has been also noted that solution preparation of antibodies or fusion protein often turns very viscous due to intermolecular interaction.

Based on the increased demand in the concentrated liquid formulation comprising fusion proteins, there is always needed to prepare stable liquid formulations comprising high concentrated pharmacologically fusion protein.

It is observed that many of fusion protein such as Etanercept, Abatacept, Belatacept, Aflibercept are formulated with phosphate buffer. However, the present invention provides an improved formulation capable to stabilize high concentration fusion protein by using histidine as buffer, stabilizer and optionally amino acid.

The formulations of the invention are useful for subcutaneous delivery to a patient in need thereof. In order to deliver maximum therapeutic benefits to patients, it is desirable that formulations for subcutaneous (SC) delivery comprise a high antibody concentration. However, high concentration of antibody, or antigen-binding fragment thereof, may contribute to other properties of the product which would be undesirable, e.g., low injectability due to increased viscosity and higher than physiological osmolality and increased aggregation.

For an example, commercial formulation of Abatacept has very high amount of sucrose around 170 mg/ml which makes the formulation very viscous. Therefore, it is preferred that an antibody product intended for SC administration balances the effects of concentration while maintaining a level of drug that will provide the highest therapeutic benefit.

An ideal product comprises a high protein concentration, low viscosity, an osmolality similar to physiological conditions, and a low level of aggregation under typical storage conditions. Increased viscosity at high protein concentration may not only make it difficult to extract the product from its container with a syringe, but also to inject the necessary dose into a patient from the syringe (syringeability). Advantageously, embodiments of the invention provide formulations that comprise a high concentration fusion protein thereof, and a viscosity level that is acceptable for subcutaneous delivery. Additionally, the formulations of the invention do not lead to high levels of aggregation.

### SUMMARY OF THE INVENTION

The present invention relates to stable liquid formulations comprising high concentrated pharmacologically active fusion protein and process for preparation of the same.

The present invention discloses a stable pharmaceutical formulation of high concentration of fusion protein molecule comprising histidine buffer, amino acid, stabilizer and surfactant, wherein the fusion protein comprises the receptor fused with constant region of Immunoglobulin.

The present invention discloses a stable pharmaceutical formulation of high concentration of fusion protein molecule comprising histidine buffer, amino acid, stabilizer and surfactant, wherein the fusion protein is a CTLA4-I_{g} molecule.

The present invention discloses a stable pharmaceutical formulation of high concentration of fusion protein molecule where the concentration of fusion protein is above 100 mg/ml.

The present invention discloses a stable pharmaceutical formulation of high concentration of a fusion protein molecule comprising histidine buffer, amino acid, surfactant and stabilizer, wherein the fusion protein is a CTLA4-Ig molecule; wherein the stabilizer is sugar which is present in lower amount than the CTLA4-Ig molecule.

In certain embodiment, the invention provides a stable formulation comprises high concentration of fusion protein and at least one amino acid and suitable excipients wherein amino acid is selected from arginine, lysine, proline, glycine, phenylalanine, aspartic acid, methionine, glutamic acid, and asparagine.

In certain embodiment, the formulations of the present invention are stable and avoid problem of precipitation of pharmacologically active fusion protein.

In an embodiment, the stable formulation of the present invention possesses advantageous properties such as low viscosity, low aggregation, and low osmolality.

In certain embodiment, the invention provides can be supplied in pre-filled syringe or auto-injector.

In an embodiment, the present invention provides a pharmaceutical formulation comprising;
a. high concentration of pharmacologically active fusion protein;
b. suitable buffer comprising L-histidine and L-histidine HCl;
c. suitable amino acid selected from arginine, lysine, glycine, proline, methionine and combination thereof;
d. suitable stabilizer;
e. suitable surfactant selected from polysorbate 20, polysorbate 80 or Poloxamer 188 and;
f. pH selected from about from 6.5 to about 7.5.

In an embodiment, the present invention provides a pharmaceutical formulation comprising;
a. high concentration of pharmacologically active CTLA4-Ig fusion protein;
b. suitable buffer comprising L-histidine and L-histidine HCl;
c. suitable amino acid selected from arginine, lysine, glycine, proline, methionine and combination thereof;
d. suitable stabilizer;
e. at least one surfactant selected from polysorbate 20, polysorbate 80 or Poloxamer 188 and;
f. pH selected from about from 6.5 to about 7.5.

In an embodiment, the present invention provides a pharmaceutical formulation comprising;
a. high concentration of pharmacologically active CTLA4-Ig fusion protein;
b. suitable buffer comprising L-histidine and L-histidine HCl;
c. suitable amino acid selected from arginine, lysine, glycine, proline, methionine and combination thereof;
d. suitable stabilizer is selected from sucrose or cyclodextrin;
e. suitable surfactant selected from polysorbate 20, polysorbate 80 or Poloxamer 188 and;
f. pH selected from about from 6.5 to about 7.5.

In an embodiment, the present invention provides a pharmaceutical formulation comprising;
a. high concentration of pharmacologically active CTLA4-Ig fusion protein;
b. suitable buffer comprising L-histidine and L-histidine HCl;
c. suitable amino acid is arginine or arginine HCl;
d. sucrose;
e. surfactant is poloxamer 188 and;
f. pH 7.3±0.2;

In an embodiment, the present invention a pharmaceutical formulation comprising;
a. high concentration of pharmacologically active CTLA4-Ig fusion protein;
b. suitable buffer comprising L-histidine and L-histidine HCl;
c. suitable amino acid is lysine or lysine HCl;
d. sucrose;
e. surfactant is poloxamer 188 and;
f. pH 7.3±0.2

In certain embodiment the sugar concentration is lower than concentration of fusion protein CTLA4-Ig. In certain embodiment the fusion protein and sugar ratio is selected from not more than 1:0.9, 1:0.8, 1:0.7, 1:0.6, or 1:0.5.

In an embodiment, the present invention provides a stable pharmaceutical formulation comprising;
a. high concentration of pharmacologically active CTLA4-Ig fusion protein;
b. suitable buffer comprising L-histidine and L-histidine HCl;
c. suitable amino acid is arginine or arginine HCl;
d. suitable amino acid is lysine or lysine HCl;
e. sucrose;
f. surfactant is poloxamer 188 and;
g. pH 7.3±0.2.

In another embodiment, the present invention provides a stable pharmaceutical formulation comprising;
a. high concentration of pharmacologically active CTLA4-Ig fusion protein;
b. suitable buffer comprising L-histidine and L-histidine HCl;
c. sucrose;
d. surfactant is Poloxamer 188 and;
e. pH 7.3±0.2.
wherein the concentration of histidine buffer is at least 150 mM.

In certain embodiment, the stable pharmaceutical formulation has kinematic viscosity of at least about 5 cPs. In certain embodiment, the formulation has viscosity of about 5 cPs to about 10 cPs.

In certain embodiment, the formulation has kinematic viscosity of about 7 cPs to about 10 cPs. In certain embodiment, the formulation has kinematic viscosity of about 8 cPs to about 10 cPs.

In preferred embodiment, the stable pharmaceutical formulation has kinematic viscosity of about 10 cPs or less.

In another preferred embodiment, the stable pharmaceutical formulation has kinematic viscosity of about 7 cPs to about 9 cPs.

In certain embodiment, the stable pharmaceutical formulation has osmolality selected from about 700 to about 900 mOsm/kg. In certain embodiment, the stable pharmaceutical formulation has osmolality selected from about 730 to about 900 mOsm/kg. In certain embodiment, the stable pharmaceutical formulation has osmolality selected from about 750 to about 900 mOsm/kg. In certain embodiment, the stable pharmaceutical formulation has osmolality selected from about 770 to about 850 mOsm/kg.

In preferred embodiment, the stable pharmaceutical formulation has osmolality of about 800±50 mOsm/kg.

In another preferred embodiment, the stable pharmaceutical formulation has osmolality of about 780 to about 825 mOsm/kg.

### DETAILED DESCRIPTION OF THE PREFERRED EMBODIMENT

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. The invention is not limited to the various embodiments given in this specification. Unless otherwise defined, all technical and scientific terms used herein have the same meaning as commonly understood by one of ordinary skill in the art to which this invention pertains. In the case of conflict, the present document, including definitions will control.

The term “fusion protein” refers to proteins created through the joining of two or more genes that originally coded for separate proteins. The fusion proteins are made using recombinant DNA techniques. Fusion protein consisting of receptor including but not limited to selected from CTLA4, TNFR, VEGF, HER-2, PCSK9 fused with constant region of immunoglobulin selected from IgG1, IgG2, IgG3 and IgG4. In addition, any modification is performed in natural amino acid to obtain desired biological activity.

The terms “CTLA4-Ig” or “CTLA4-Ig molecule” or “CTLA4Ig molecule” are used interchangeably and refer to a protein molecule that comprises at least a polypeptide having a CTLA4 extracellular domain or portion thereof and an immunoglobulin constant region or portion thereof. The extracellular domain and the immunoglobulin constant region can be wild-type, or mutant or modified, and mammalian, including human or mouse. The polypeptide can further comprise additional protein domains. A CTLA4-Ig molecule can also refer to multimer forms of the polypeptide, such as dimers, tetramers, and hexamers. A CTLA4-Ig molecule is also capable of binding to CD80 and/or CD86. In certain embodiment, the CTLA4-Ig is Abatacept.

The term “B7-1” refers to CD80; the term “B7-2” refers CD86; and the term “B7” refers to both B7-1 and B7-2 (CD80 and CD86). The term “B7-1-Ig” or “B7-1Ig” refers to CD80-Ig; the term “B7-2-Ig” or “B7-2-Ig” refers CD86-Ig.

As used throughout the specification and in the appended claims, the singular forms “a,” “an,” and “the” include the plural reference unless the context clearly dictates otherwise.

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or formulation. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

The term “around,” “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

The term used “Size variants” refers to LMW, HMW or aggregates.

The term used “Low molecular weight (LMW) species” which is a protein backbone-truncated fragments & considered as product-related impurities that contribute to the size heterogeneity of antibody. LMW species often have low or substantially reduced activity relative to the monomeric form of the antibody and can lead to immunogenicity or potentially impact pharmacokinetic properties in vivo. As a result, LMW species are considered critical quality attributes that are routinely monitored during drug development and as part of release testing of purified drug product during manufacturing.

The term used “high molecular weight or HMW” is product-related impurities that contribute to the size heterogeneity of antibody products. The formation of HMW species within a therapeutic antibody drug product as a result of protein aggregation can potentially compromise both drug efficacy and safety (e.g., eliciting unwanted immunogenic response). HMW is considered critical quality attributes that are routinely monitored during drug development and as part of release testing of purified drug product during manufacturing.

The term used “aggregates” are classified based on types of interactions and solubility. Soluble aggregates are invisible particles and cannot be removed with a filter. Insoluble aggregates can be removed by filtration and are often visible to the human eye. Both types of aggregates cause problems in biopharma development. Covalent aggregates arise from the formation of a covalent bond between multiple monomers of a given peptide. Disulfide bond formation of free thiols is a common mechanism for covalent aggregation. Oxidation of tyrosine residues can lead to formation of bityrosine which often results in aggregation. Reversible protein aggregation typically results from weaker protein interactions they include dimers, trimers, multimers among others.

A “surfactant” is a surface-active molecule containing both a hydrophobic portion (e.g., alkyl chain) and a hydrophilic portion (e.g., carboxyl and carboxylate groups). Surfactant may be added to the formulations of the invention. Surfactants suitable for use in the formulations of the present invention include, but are not limited to, polysorbates (e.g., polysorbates 20 or 80); poloxamers (e.g., poloxamer 188).

The term “pharmaceutical” as used herein with reference to a composition, e.g., an aqueous formulation, that it is useful for treating a disease or disorder.

The term “pharmaceutical formulation” refers to preparations which are in such a form as to permit the biological activity of the active ingredients to be effective, and therefore may be administered to a subject for therapeutic use.

A “stable” formulation is one in which the protein therein essentially retains its physical stability and/or chemical stability and/or biological activity upon storage. Various analytical techniques for measuring protein stability are available in the art for example. In one embodiment, the stability of the protein is determined according to the percentage of monomer protein in the solution, with a low percentage of degraded (e.g., fragmented) and/or aggregated protein.

The term “stabilizer” refers to excipients that are used to maintain the desirable properties of the product until it is consumed by the patient. The stabilizers may be sugars, polymers, salts etc.

Stabilizer can stabilize the fusion protein to for at least 2 weeks, at least 4 weeks, at least 1 month, at least 2 months, at least 3 months, at least 6 months, at least 8 months, at least 12 months based on the selection of temperature and sugar.

The term “sugar” refers to monosaccharides, disaccharides, oligosaccharides and polysaccharides. Examples include sucrose, trehalose, dextrose, maltose, sorbitol, mannitol, cyclodextrin and others. In most preferred embodiment, the sugar is sucrose.

The term “viscosity” refers to the resistance of a liquid formulation to flow, such as when injected through a syringe needle during administration to a patient. Viscosity of a protein solution depends on the nature of the individual and protein-protein interaction (PPI). Both of the individual characteristics, such as particle size and shape, as well as the pair interactions can be influenced by components in the formulation. As a result, it is often desirable to reduce viscosity values so that formulations are suitable for a particular application or process (i.e.: injection). Increased viscosity at high protein concentration may not only make it difficult to extract the product from its container with a syringe, but also to inject the necessary dose into a patient from the syringe (syringeability parameters). Increased viscosity also leads to more challenging in handling product during manufacturing (such as filtration and filling).

Hence, it is desirable to keep viscosity low in formulation as it increases glide force and break loose force of syringe. Furthermore, it is known that high viscosity causes severe pain to patient during injection. In addition, high viscosity causes loss of product due to its tendency to bind to container closure.

It is noted that commercial formulation of CTLA4-IgG1 (Abatacept) contains high sucrose concentration i.e., 170 mg/ml which makes the formulation very viscous.

It is understood that the value of viscosity is dependent on the conditions under which the measurement was taken, such as temperature, the rate of shear and the shear stress employed. The apparent viscosity is defined as the ratio of the shear stress to the rate of shear applied. There are number of alternative methods for measuring apparent viscosity. For example, viscosity can be tested by a suitable cone and plate, parallel plate or other type of viscometer or rheometer.

The term “osmolality” refers to a measure of solute concentration, defined as the number of mmole of solute per kg of solution. A desired level of osmolality can be achieved by the addition of one or more stabilizer such as a sugar or polyol including sorbitol, mannitol, dextrose, trehalose, maltose, mannose and/or sucrose.

The present invention provides stable and improved liquid formulations which can optionally be lyophilized, comprising of high concentration of therapeutic protein(s), preferably fusion protein, in suitable buffer(s), one or more suitable amino acid, and other excipients which are optionally selected from suitable surfactants and stabilizer or tonicity agents. The stable formulation provides desirable viscosity & prevents formation of aggregates of protein.

In certain embodiment, the novel formulations of the present invention are suitable for subcutaneous administration. The novel formulations of the present invention are stable and avoid precipitation of pharmacologically active fusion protein.

In certain embodiment, the present invention is stable at room temperature. In certain embodiment, the novel formulations of the present invention are stable at 2° C. to 8° C.

In certain embodiment, the formulation provides stability of fusion protein for at least two weeks. In an embodiment, the formulation is stable for at least 1 month, 3 months, 6 months, 10 months, 1 year or 2 years.

In certain embodiment, the novel formulations of the present invention are stable at 40° C.

In certain embodiment, the formulation provides stability of fusion protein for at least two weeks to one month.

In certain embodiment, the novel formulations of the present invention comprise high concentration of pharmacologically active fusion protein, histidine buffer, amino acid, sugar and surfactant.

In certain embodiment, the pharmacology active fusion protein binds with CD80/86 to block T cell activation for the treatment of arthritis. In certain embodiment, the pharmacology fusion protein is CTLA4-IgG1 (Abatacept).

In an embodiment, the novel formulations of the present invention are mainly useful for the treatment of, but not limited to rheumatoid arthritis (RA), Juvenile Idiopathic Arthritis (pJIA), graft versus host disease (GVHD) and psoriatic arthritis (PsA).

In embodiment, the pharmacological active fusion protein is present in high concentration at least 100 mg/ml. In certain embodiment, the pharmacological fusion protein is present in high concentration selected from about 100 mg/ml to about 200 mg/ml. In certain embodiment, the pharmacological fusion protein is present in high concentration of about 125 mg/ml. The skilled person can adjust the concentration of protein for an example 87.5 mg/0.7 mL and 50 mg/0.4 mL.

In certain embodiment, the pharmacological active fusion protein is present in high concentration selected from 125 mg/ml.

In certain embodiment, the pharmacological active fusion protein is present in high concentration selected from 87.5 mg/0.7 mL.

In certain embodiment, the pharmacological active fusion protein is present in high concentration selected from 50 mg/0.4 mL. In certain embodiment, the invention provides a novel stable formulation comprises a suitable buffer selected from phosphate, citrate, phosphate-citrate, histidine and acetate and salt thereof. However, the present invention found the histidine is most suitable buffer for stabilizing fusion protein.

In certain embodiment, the invention provides a novel stable formulation comprises more than one buffer.

The present invention provides novel liquid formulations comprising high concentrated pharmacologically active fusion protein by using histidine as a buffer. The present applicant found that histidine has suitable buffering property for fusion protein and provides better stability in combination with sucrose and/or amino acid and surfactant.

In certain embodiment, the histidine buffer is prepared by L-histidine and L-histidine HCl.

In certain embodiment, histidine can also be used in its salt form such as histidine hydrochloride.

In certain embodiment, the buffer concentration is at least about 1 mg/ml. In certain embodiment, the buffer concentration is in the range of about 1 mg/ml to about 10 mg/ml. In certain embodiment, the buffer concentration is in the range of about 1 mg/ml to about 5 mg/ml. In certain embodiment, the buffer concentration is about 4 mg/ml. In certain embodiment, the buffer concentration is about 4.2 mg/ml.

In certain embodiment, the buffer is concentration is at least about 50 mM. In certain embodiment, the buffer is concentration is in the range of about 50 mM to about 200 mM.

In certain embodiment, the buffer is concentration is in the range of about 20 mM, about 25 mM, about 30 mM, about 35 mM, about 40 mM, about 45 mM, about 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, about 140 mM to about 150 mM, about 150 mM to about 160 mM, about 160 mM to about 170 mM, about 170 mM to about 180 mM, about 180 mM to about 190 mM, about 195 mM, about 200 mM, about 203 mM and about 205 mM.

In an embodiment, the concentration of histidine buffer is more than 100 mM. In an embodiment, the concentration of histidine buffer is in the range of about 125 mM, about 140 mM, about 150 mM, about 170 mM, about 190 mM, about 200 mM, about 203 mM and about 205 mM.

In an embodiment, the concentration of histidine buffer is about 150 mM.

In another embodiment, the concentration of histidine buffer is below about 100 mM. in certain embodiment the concentration of histidine buffer is about 50 mM, about 60 mM, about 70 mM, about 80 mM and about 90 mM.

In another embodiment, the formulation comprising the buffer concentration is about 200 mM to about 205 mM. In another preferred embodiment the buffer concentration is about 203 mM. In such embodiment, the formulation reduced or does not contain amino acid the formulation doesn't comprise or have reduced amount of amino acid when buffer concentration is about 200 mM or above.

In certain embodiment, amino acids are selected from arginine, lysine, methionine, glycine, proline, asparagine, phenylalanine & suitable salt thereof. In certain embodiment, the lysine, and arginine can be used in its salt form such as lysine hydrochloride, or arginine hydrochloride.

In preferred embodiment, the formulation does not comprise histidine as amino acid while the present formulation only uses histidine as buffer. In another preferred embodiment the formulation comprising histidine buffer and amino acid selected from arginine, lysine, methionine, glycine, proline, asparagine, phenylalanine & suitable salt thereof along with suitable stabilizer and surfactant. The formulation is essentially free of histidine as amino acid.

In certain embodiment, the amino acid present in the concentration of at least about 1 mg/ml. In certain embodiment, the amino acid present in the range of about 1 mg/ml to about 75 mg/ml. In certain embodiment, the amino acid present in the range of about 5 mg/ml to about 50 mg/ml. In certain embodiment, the amino acid present in range of about 9 mg/ml to about 50 mg/ml. In certain embodiment, the amino acid present in range of about 9 mg/ml to about 20 mg/ml.

In certain embodiment, the amino acid arginine presents in the concentration from about 7 mg/ml to about 25 mg/ml. In certain embodiment, the amino acid arginine presents in the concentration from about 15 mg/ml to about 21 mg/ml.

In another aspects, formulation is optimized with low concentration of arginine. In certain embodiment, the amino acid arginine presents in the concentration from about 7 mg/ml to about 14 mg/ml. In preferred embodiment, the arginine concentration is about 9 mg/ml to about 11 mg/ml.

In another preferred embodiment, the arginine concentration is about 10.5 mg/ml to about 11 mg/ml.

In certain embodiment, the amino acid lysine presents in the concentration from about 7 mg/ml to about 20 mg/ml. In certain embodiment, the amino acid lysine presents in the concentration from about 11 mg/ml to about 13.70 mg/ml.

In another aspects, formulation can be optimized with low concentration of lysine.

In certain embodiment, the amino acid lysine presents in the concentration from about 7 mg/ml to about 13 mg/ml. In preferred embodiment, the lysine concentration is about 9 mg/ml to about 11 mg/ml. In another preferred embodiment, the lysine concentration is about 9.3 mg/ml to about 10 mg/ml.

In another embodiment, the formulation comprises arginine or lysine or both.

In certain embodiment, the amino acid is present in the concentration of at least about 50 mM. In certain embodiment, the amino acid is present in the range of about 50 mM to about 60 mM, about 60 mM to about 70 mM, about 70 mM to about 80 mM, about 80 mM to about 90 mM, about 90 mM to about 100 mM, about 100 mM to about 110 mM, about 110 mM to about 120 mM, about 120 mM to about 130 mM, about 130 mM to about 140 mM, and about 140 mM to about 150 mM.

In certain embodiment, the amino acid arginine presents in the concentration more than about 50 mM. In certain embodiment, the amino acid arginine presents in the concentration from about 50 mM to about 150 mM. In certain embodiment, the amino acid arginine presents in the concentration from about 80 mM to about 125 mM. In preferred embodiment, the amino acid arginine presents in the concentration from about 100 mM to about 110 mM.

In another aspects, formulation can be optimized with low concentration of arginine. In certain embodiment, the amino acid arginine presents in the concentration from about 25 mM to about 50 mM. In preferred embodiment, the arginine concentration is about 30 mM to about 50 mM. In another preferred embodiment, the arginine concentration is about 40 mM to about 50 mM.

In certain embodiment, the amino acid lysine presents in the concentration more than about 50 mM. In certain embodiment, the amino acid lysine presents in the concentration from about 50 mM to about 100 mM. In preferred embodiment, the amino acid lysine presents in the concentration from about 75 mM to about 80 mM.

In another aspects, formulation can be optimized with low concentration of lysine. In certain embodiment the amino acid lysine presents in the concentration from about 25 mM to about 50 mM. In preferred embodiment, the lysine concentration is about 30 mM to about 50 mM. In another preferred embodiment, the lysine concentration is about 40 mM to about 50 mM. In certain embodiment, the stabilizers selected from sugars.

In certain embodiment, the stabilizer is selected from sucrose or cyclodextrin.

In preferred embodiment, the stabilizer is sucrose.

In certain embodiment, the sugar concentration is lower than concentration of fusion protein. In certain embodiment, the fusion protein and sugar ratio is selected from not more than 1:0.9, 1:0.8, 1:0.7, 1:0.6, or 1:0.5.

In certain embodiment, the sucrose concentration is lower than concentration of fusion protein. In certain embodiment, the fusion protein and sucrose ratio is selected from not more than 1:0.9, 1:0.8, 1:0.7, 1:0.6, or 1:0.5.

In certain embodiment, the sucrose is in the concentration of at least about 50 g/L. In certain embodiment, the sucrose is present in the range of about 50 g/L to about 60 g/L, about 60 g/L to about 70 g/L, about 70 g/L to about 80 g/L, about 80 g/L to about 90 g/L, about 90 g/L to about 100 g/L, and about 110 g/L to about 120 g/L.

In an embodiment, the sucrose is present in the concentration of about 100 g/L, about 101 g/L, about 102 g/L, about 103 g/L, about 104 g/L, about 105 g/L, about 106 g/L, about 107 g/L, about 108 g/L, about 109 g/L and about 110 g/L.

In certain embodiment, the surfactant is selected from polysorbate and poloxamer 188. In certain embodiment, the surfactant is selected from different grades of polysorbate such as but not limited to polysorbate 20, polysorbate 80, poloxamer 188 or mixture thereof can be used. In certain embodiment, the surfactant is poloxamer 188.

In certain embodiment, the surfactant is present in the concentration of at least about 1 mg/ml. In certain embodiment, the surfactant is present in amount of about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, and about 15 mg/ml.

In preferred embodiment, the surfactant is present in concentration of about 8 mg/ml.

In certain embodiment, the novel formulations of the present invention have of about pH 6.0 to about pH 7.5. In certain embodiment, the novel formulations of the present invention have of about pH 6.5 to about pH 7.5. In an embodiment, the novel formulations of the present invention have of about pH 6.5, about pH 6.6, about pH 6.7, about pH 6.8, about pH 6.9, about pH 7.0, about pH 7.1, about pH 7.2, about pH 7.3, about pH 7.4, and about pH 7.5.

In preferred embodiment the pH is 7.3±0.2.

In certain embodiment, the novel formulations have viscosity of at least about 5 cps. In certain embodiment, the formulation has viscosity of about 5 cps to about 15 cps. In certain embodiment, the formulation has viscosity of about 7 cps to about 15 cps. In certain embodiment, the formulation has viscosity of about 9 cps to about 15 cps.

In one aspect of such embodiment, the viscosity of the novel formulation is about 5 cps, about 6 cps, about 7 cps, about 8 cps, about 9 cps, about 10 cps, about 1 lcps, about 12 cps, about 13 cps, about 14 cps and about 15 cps.

In preferred embodiment, the novel formulation has kinematic viscosity of about 10 cPs or less.

In another preferred embodiment, the novel formulation has kinematic viscosity of about 7 cPs to about 9 cPs.

In certain embodiment, the present invention discloses a desirable viscosity about 7 cps to about 9 cps when formulated fusion protein CTLA4-Ig in histidine buffer with arginine or lysine in combination with sucrose and surfactant.

In another embodiment, the present invention discloses a desirable viscosity about 8 cps to about 10 cps when formulated fusion protein CTLA4-Ig in histidine buffer with arginine or lysine in combination with sucrose and surfactant.

In certain embodiment, the stable pharmaceutical novel liquid formulations have osmolality selected from about 700 to about 900 mOsm/kg. In certain embodiment, the stable pharmaceutical novel liquid formulations have osmolality selected from about 730 to about 900 mOsm/kg. In certain embodiment, the stable pharmaceutical novel liquid formulations have osmolality selected from about 750 to about 900 mOsm/kg. In certain embodiment, the stable pharmaceutical novel liquid formulations have osmolality selected from about 770 to about 900 mOsm/kg.

In one aspect of such embodiment, the osmolality of the novel formulation is about 700 mOsm/kg, about 710 mOsm/kg, about 720 mOsm/kg, about 730 mOsm/kg, about 740 mOsm/kg, about 750 mOsm/kg, about 760 mOsm/kg, about 770 mOsm/kg, about 780 mOsm/kg, about 790 mOsm/kg, about 800 mOsm/kg, about 810 mOsm/kg, about 820 mOsm/kg, about 830 mOsm/kg, about 840 mOsm/kg, about 850 mOsm/kg, about 860 mOsm/kg, about 870 mOsm/kg, about 890 mOsm/kg, and about 900 mOsm/kg.

In certain aspect, the present invention discloses a desirable viscosity about 750 mOsm/Kg to about 850 mOsm/Kg when formulated fusion protein CTLA4-Ig in histidine buffer with arginine or lysine in combination with sucrose and surfactant.

In preferred embodiment, the novel formulation has osmolality of about 800±50 mOsm/kg.

In another preferred embodiment, the novel formulation has osmolality of about 780 to about 825 mOsm/kg

In another embodiment, the present invention discloses a desirable viscosity about 780 mOsm/Kg to about 825 mOsm/Kg when formulated fusion protein CTLA4-Ig in histidine buffer with arginine or lysine in combination with sucrose and surfactant.

In an embodiment, the present invention discloses a stable pharmaceutical formulation of a fusion protein molecule comprising histidine buffer, amino acid, stabilizer and surfactant, wherein the fusion protein comprises the receptor fused with constant region of Immunoglobulin.

In an embodiment, the present invention discloses a stable pharmaceutical formulation of a fusion protein molecule comprising histidine buffer, amino acid, stabilizer and surfactant, wherein the fusion protein is a CTLA4-Ig molecule.

In an embodiment, the present invention discloses a stable pharmaceutical formulation of a fusion protein molecule comprising buffer, amino acid, surfactant and stabilizer, wherein the fusion protein is a CTLA4-Ig molecule; wherein the stabilizer is sugar which is present in lower amount than the CTLA4-IgG.

In certain embodiment, the invention provides a novel stable formulation comprises high concentration of fusion protein and at least one suitable aggregation inhibitor and suitable excipients wherein the suitable aggregation inhibitor is amino acid selected from arginine, lysine, proline, glycine, phenylalanine, aspartic acid, methionine, glutamic acid, and asparagine.

In an embodiment, the present invention provides a stable pharmaceutical formulation comprising;
a. high concentration of pharmacologically active fusion protein;
b. suitable buffer L-histidine and L-histidine HCl;
c. suitable amino acid selected from arginine, lysine, glycine, proline, methionine and combination thereof;
d. suitable stabilizer;
e. suitable surfactant selected from polysorbate 20, polysorbate 80 or Poloxamer 188 and;
f. pH selected from about from 6.5 to about 7.5.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid selected from arginine, lysine, glycine, proline, methionine and combination thereof;
d. suitable stabilizer;
e. at least one surfactant selected from polysorbate 20, polysorbate 80 or Poloxamer 188 and;
f. pH selected from about from 6.5 to about 7.5.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid selected from arginine, lysine, glycine, proline, methionine and combination thereof;
d. suitable stabilizer is sucrose;
e. suitable surfactant selected from polysorbate 20, polysorbate 80 or Poloxamer 188 and;
f. pH selected from about from 6.5 to about 7.5.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid selected from arginine, lysine, glycine, proline, methionine and combination thereof;
d. suitable stabilizer is cyclodextrin;
e. suitable surfactant selected from polysorbate 20, polysorbate 80 or Poloxamer 188 and;
f. pH selected from about from 6.5 to about 7.5.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid is arginine or arginine HCl;
d. sucrose;
e. poloxamer 188 and;
f. pH 7.3±0.2;

In an embodiment, the present invention provides a stable pharmaceutical formulation comprising;
a. high concentration of pharmacologically active CTLA4-Ig fusion protein;
b. suitable buffer comprising L-histidine and L-histidine HCl;
c. suitable amino acid is lysine or lysine HCl;
d. sucrose;
e. poloxamer 188 and;
f. pH 7.3±0.2

In certain embodiment the sucrose concentration is lower than concentration of fusion protein. In certain embodiment the fusion protein and sucrose ratio is selected from not more than 1:0.9, 1:0.8, 1:0.7, 1:0.6, or 1:0.5.

In an embodiment, the present invention provides a stable pharmaceutical formulation comprising;
a. high concentration of pharmacologically active CTLA4 Ig fusion protein;
b. suitable buffer L-histidine and L-histidine HCl;
c. suitable amino acid is arginine or arginine HCl;
d. suitable amino acid is lysine or lysine HCl;
e. sucrose;
f. poloxamer 188 and;
g. pH 7.3±0.2

The pharmacologically active fusion protein which binds to CD80 and CD86 molecule is abatacept or belatacept.

In another aspect of such embodiment the formulation has high monomer and low aggregation or HMW % and LMW %.

In certain embodiment the formulation provides HMW % below 10% or 9% or 8% or 7% or 6% or 5% or 4% or 3% or 2% or 1% when stored at 25° C. for 2 weeks analysed by SEC-HPLC or CE-SDS.

In certain embodiment the formulation provides LMW % below 10% or 9% or 8% or 7% or 6% or 5% or 4% or 3% or 2% or 1% when stored at 25° C. for 2 weeks analysed by SEC-HPLC or CE-SDS.

In certain embodiment the formulation provides HMW % below 10% or 9% or 8% or 7% or 6% or 5% or 4% or 3% or 2% or 1% when stored at 25° C. for 4 weeks analysed by SEC-HPLC or CE-SDS.

In certain embodiment the formulation provides LMW % below 10% or 9% or 8% or 7% or 6% or 5% or 4% or 3% or 2% or 1% when stored at 25° C. for 4 weeks analysed by SEC-HPLC or CE-SDS.

In certain embodiment the formulation provides monomer more than 90%. In certain embodiment the formulation provides monomer more than 95%, 96%, 97%, 98% analysed by SEC-HPLC or CE-SDS.

In certain embodiment the formulation is stable for at least 2 weeks at 25° C. In certain embodiment the formulation is stable for at least 4 weeks at 25° C. In certain embodiment the formulation is stable for at least 8 weeks at 25° C. In certain embodiment the formulation is stable for at least 12 weeks at 25° C. In certain embodiment the formulation is stable for at least 16 weeks at 25° C. In certain embodiment the formulation is stable for at least 20 weeks at 25° C. In certain embodiment the formulation is stable for at least 24 weeks at 25° C.

In certain embodiment the formulation is stable for at least one weeks at 30° C. In certain embodiment the formulation is stable for at least 2 weeks at 30° C. In certain embodiment the formulation is stable for at least 8 weeks at 2° C. to 8° C.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid is arginine or arginine HCl;
d. sucrose;
e. polysorbate 20 and;
f. pH 7.3±0.2;

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid is lysine or lysine HCl;
d. sucrose;
e. polysorbate 20 and;
f. pH 7.3±0.2

The present invention discloses that use of amino acid can be reduced or avoided to prepare stable formulation when histidine buffer is used in high concentration more than about 150 mM, about 160 mM, about 170 mM, about 180 mM, about 190 mM and about 200 mM.

In another embodiment, the present invention provides a stable pharmaceutical formulation comprising;
a. high concentration of pharmacologically active CTLA4 Ig fusion protein;
b. suitable buffer L-histidine and L-histidine HCl;
c. Sucrose;
d. Optionally suitable aggregation inhibitor selected from arginine, glycine or lysine and salt thereof;
e. poloxamer 188 and;
f. pH selected from about from 7.1 to about 7.4;
wherein the histidine buffer concentration is about 150 mM to about 200 mM

In another embodiment, the present invention provides a stable pharmaceutical formulation comprising;
a. high concentration of pharmacologically active CTLA4 Ig fusion protein;
b. suitable buffer L-histidine and L-histidine HCl;
c. suitable amino acid is arginine, or its salt selected from arginine HCl, arginine citrate, arginine succinate, arginine phosphate, arginine sulphate;
d. sucrose;
e. poloxamer 188;
f. pH selected from about from 7.1 to about 7.4.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of about 25 mM to 80 mM;
c. suitable amino acid is arginine or arginine HCl in an amount of about 50 mM to 125 mM;
d. sucrose in an amount of about 100 g/L to 130 g/L;
e. Poloxamer 188 in an amount of about 1 g/L to 10 g/L and;
f. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of about 25 mM;
c. suitable amino acid is arginine or arginine HCl in an amount of about 125 mM;
d. sucrose in an amount of about 110 g/L;
e. Poloxamer 188 in an amount of about 8 g/L and;
f. pH 7.3±0.2;

In such embodiment the viscosity is 8.41 cPs and osmolality is 856 mOsm/kg

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of about 100 mM to 200 mM;
c. suitable amino acid is arginine or arginine HCl in an amount of about 10 mM to 80 mM;
d. sucrose in an amount of about 100 g/L to 110 g/L;
e. Poloxamer 188 in an amount of about 1 g/L to 10 g/L and;
f. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of 150 mM;
c. suitable amino acid arginine or arginine HCl in an amount of 50 mM;
d. sucrose in an amount of 110 g/L;
e. Poloxamer 188 in an amount of 8 g/L and;
f. pH 7.3±0.2.

In such embodiment the viscosity is 8.09 cPs and osmolality is 785 mOsm/kg

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of about 100 mM to 200 mM;
c. suitable amino acid is lysine or lysine HCl in an amount of about 10 mM to 80 mM;
d. sucrose in an amount of about 100 g/L to 110 g/L;
e. Poloxamer 188 in an amount of about 1 g/L to 10 g/L and;
f. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of 150 mM;
c. suitable amino acid lysine or lysine HCl in an amount of 50 mM;
d. sucrose in an amount of 110 g/L;
e. poloxamer 188 in an amount of 8 g/L and;
f. pH 7.3±0.2.

In such embodiment the viscosity is 8.74 cPs and osmolality is 818 mOsm/kg.

In an embodiment, the present invention provides a stable pharmaceutical formulation comprising;
a. high concentration of pharmacologically active CTLA4-Ig fusion protein;
b. suitable buffer L-histidine and L-histidine HCl;
c. suitable amino acid is arginine or arginine HCl;
d. suitable amino acid is lysine or lysine HCl;
e. sucrose;
f. poloxamer 188 and;
g. pH 7.3±0.2.

In an embodiment, the present invention provides a stable pharmaceutical formulation comprising;
a. high concentration of pharmacologically active CTLA4 Ig fusion protein in an amount of about 125 mg/ml;
b. buffer L-histidine and L-histidine HCl in an amount of about 10 mM to 80 mM;
c. suitable amino acid is arginine or arginine HCl in an amount of about 50 mM to 140 mM;
d. suitable amino acid is lysine or lysine HCl in an amount of about 20 mM to 100 mM;
e. sucrose in an amount of about 100 g/L to 110 g/L;
f. poloxamer 188 in an amount of about 1 g/L to 10 g/L and;
g. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of 25 mM;
c. suitable amino acid is arginine or arginine HCl in an amount of 100 mM;
d. suitable amino acid is lysine or lysine HCl in an amount of 75 mM;
e. sucrose in an amount of 100 g/L;
f. poloxamer 188 in an amount of 8 g/L and;
g. pH 7.3±0.2;

In such embodiment the viscosity is 7.51 cPs and osmolality is 846 mOsm/kg.

In an embodiment, the present invention provides a stable pharmaceutical formulation comprising;
a. high concentration of pharmacologically active CTLA4 Ig fusion protein in an amount of =125 mg/ml;
b. buffer L-histidine and L-histidine HCl in an amount of about 10 mM to 100 mM;
c. suitable amino acid is arginine or arginine HCl in an amount of about 50 mM to 100 mM;
d. suitable amino acid is lysine or lysine HCl in an amount of about 50 mM to 100 mM;
e. sucrose in an amount of about 100 g/L to 110 g/L;
f. poloxamer 188 in an amount of about 1 g/L to 10 g/L and;
g. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in an amount of 100 mM;
c. suitable amino acid is arginine or arginine HCl in an amount of 50 mM;
d. suitable amino acid is lysine or lysine HCl in an amount of 50 mM;
e. sucrose in an amount of 110 g/L;
f. poloxamer 188 in an amount of 8 g/L and;
g. pH 7.3±0.2.

In an embodiment, the present invention provides a stable pharmaceutical formulation comprising;
a. high concentration of pharmacologically active CTLA4 Ig fusion protein;
b. suitable buffer L-histidine and L-histidine HCl;
c. sucrose;
d. poloxamer 188 and;
e. pH 7.3±0.2.

In one aspect such embodiment, the formulation is free of amino acid or suitable salt thereof.

In an embodiment, the present invention provides a stable pharmaceutical formulation comprising;
a. high concentration of pharmacologically active CTLA4 Ig fusion protein in an amount of about 125 mg/ml;
b. buffer L-histidine and L-histidine HCl in amount of about 150 mM to about 200 mM;
c. sucrose in an amount of about 100 g/L to 110 g/L;
d. poloxamer 188 in an amount of about 8 g/L and;
e. pH 7.3±0.2.

In an embodiment, the present invention provides a stable pharmaceutical formulation comprising;
a. high concentration of pharmacologically active CTLA4 Ig fusion protein in an amount of 125 mg/ml;
b. buffer L-histidine and L-histidine HCl in amount of 200 mM;
c. sucrose in an amount of 110 g/L;
d. poloxamer 188 in an amount of 8 g/L and;
e. pH 7.3±0.2; In such embodiment the viscosity is 7.84 cPs and osmolality is 736 mOsm/kg.

In an embodiment the present formulation with histidine buffer comprises reduced amount of low molecular weight (LMW) impurities compared to fusion protein formulated with phosphate buffer.

The present invention provides examples below for illustrative purpose and scope of invention is not limited with illustrative examples.

All below examples are performed at 25° C.

### Example 1

CTLA-4 IgG1 fusion protein injectable formulation was formulated with 125 mg/mL concentration for subcutaneous administration.

Formulation (F1) was prepared with addition of 20 mM of Phosphate buffer, 110 g/L Sucrose, 150 mM Arginine HCl and Poloxamer (8 mg/mL) at pH 7.3±0.2. The DS solution was filtrated with 0.2 μm filter to get the filtrated solution and filled 1 mL of filtered solution in 1 mL glass PFS. The composition of formulation (F1) is given below:

| Compositions | mg/mL |
|---|---|
| CTLA-4 IgG1 fusion protein | 125 |
| Disodium monophosphate Heptahydrate | 2.14 |
| monobasic sodium phosphate monohydrate | 0.28 |
| Sucrose | 110 |
| L-Arginine HCl (150 mM) | 31.6 |
| Poloxamer 188 | 8 |

| | Results | |
|---|---|---|
| Analytical tests | 0 Day | 14 Day |
| % High molecular weight (HMW) species by SEC | 0.43 | 2.38 |
| % Low molecular weight (LMW) species by SEC | 0.40 | 1.17 |

F1 shows highest HMW % & LMW % on Day 14 in comparison to other formulations F3 to F7 (Histidine buffer). It is also important to note that F1 formulation shows high HMW % and LMW % even on day 0 in comparison to other formulations F3 to F7 (Histidine buffer).

### Example 2

CTLA-4 IgG1 fusion protein injectable formulation was formulated with 125 mg/mL concentration for subcutaneous administration.

Formulation (F2) was prepared with addition of 20 mM of Phosphate buffer, 110 g/L Sucrose, 150 mM Lysine HCl and Poloxamer (8 mg/mL) at pH 7.3±0.2. The DS solution was filtrated with 0.2 μm filter to get the filtrated solution and filled 1 mL of filtered solution in 1 mL glass PFS. The composition of formulation (F2) is given below:

| Compositions | mg/mL |
|---|---|
| CTLA-4 IgG1 fusion protein | 125 |
| Disodium monophosphate Heptahydrate | 2.14 |
| monobasic sodium phosphate monohydrate | 0.28 |
| Sucrose | 110 |
| L-Lysine HCl (150 mM) | 27.4 |
| Poloxamer 188 | 8 |

| | Results | |
|---|---|---|
| Analytical tests | 0 Day | 14 Day |
| % High molecular weight (HMW) species by SEC | 0.39 | 1.74 |
| % Low molecular weight (LMW) species by SEC | 0.36 | 1.38 |

F2 shows highest HMW % & LMW % on Day 14 in comparison to other formulations F3 to F7 (Histidine buffer). It is also important to note that F2 formulation shows high HMW % and LMW % even on day 0 in comparison to other formulations F3 to F7 (Histidine buffer).

### Example 3

CTLA-4 IgG1 fusion protein injectable formulation was formulated with 125 mg/mL concentration for subcutaneous administration.

Formulation (F3) was prepared with addition of 25 mM of Histidine buffer, 125 g/L of Sucrose, 125 mM of Arginine HCl, and Poloxamer (8 mg/mL) at pH 7.3±0.2. The bulk solution was filtrated with 0.2 μm filter to get the filtrated solution and filled 1 mL of filtered solution in 1 mL glass PFS. The composition of formulation (F3) is given below:

| Compositions | mg/mL |
|---|---|
| CTLA-4 IgG1 fusion protein | 125 |
| L - Histidine (22 mM) | 3.5 |
| L - Histidine HCl monohydrate (3 mM) | 0.7 |
| Sucrose (110 g/L) | 110 |
| Arginine HCl (125 mM) | 26.33 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.19 | 1.61 | 2.96 |
| % Low molecular weight (LMW) species by SEC | 0.40 | 0.8 | 1.5 |

Comparison with S01 (INN PFS) where drug substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.37 | 1.38 | 1.91 |
| % Low molecular weight (LMW) species by SEC | 0.38 | 4.05 | 8.95 |

F3 has shown that HMW % need to be controlled but still able to control LMW % effectively compared to F1, F2 and S01 formulation. Further S01 shows very high LMW % on day 28. Hence it shows that histidine controls LMW % effectively.

F3 viscosity is 8.41 cPs and osmolality is 856 mOsm/kg.

### Example 4

CTLA-4 IgG1 fusion protein injectable formulation was formulated with 125 mg/mL concentration for subcutaneous administration.

Formulation (F4) was prepared with addition of 25 mM of Histidine buffer, 100 g/L of Sucrose, 75 mM of Lysine HCl, 100 mM of Arginine HCl and Poloxamer (8 mg/mL) at pH 7.3±0.2. The bulk solution was filtrated with 0.2 μm filter to get the filtrated solution and filled 1 mL of filtered solution in 1 mL glass PFS. The composition of formulation (F4) is given below:

| Compositions | mg/mL |
|---|---|
| CTLA-4 IgG1 fusion protein | 125 |
| L - Histidine (22 mM) | 3.5 |
| L - Histidine HCl monohydrate (3 mM) | 0.7 |
| Sucrose (300 mM) | 100 |
| L-Lysine (75 mM) | 13.70 |
| L-Arginine (100 mM) | 21.07 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.13 | 1.89 | 3.54 |
| % Low molecular weight (LMW) species by | 0.34 | 0.79 | 1.39 |
| CE-SDS | | | |

Comparison with S01 (INN PFS) where ding substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.37 | 1.38 | 1.91 |
| % Low molecular weight (LMW) species by SEC | 0.38 | 4.05 | 8.95 |

F4 has shown that HMW % need to be controlled but still able to control LMW % effectively compared to F1, F2 and S01 formulation. Further S01 shows very high LMW % on day 28. Hence it shows that histidine buffer is very effective controls LMW % effectively.

F4 viscosity is 7.51 cPs and osmolality is 846 mOsm/kg.

### Example 5

CTLA-4 IgG1 fusion protein injectable formulation was formulated with 125 mg/mL concentration for subcutaneous administration.

Formulation was prepared (F5) with addition of 200 mM of Histidine buffer, 110 g/L of Sucrose and Poloxamer (8 mg/mL) at pH 7.3±0.2. The bulk solution was filtrated with 0.2 μm filter to get the filtrated solution and filled 1 mL of filtered solution in 1 mL glass PFS. The composition of formulation (F5) is given below:

| Compositions | mg/mL |
|---|---|
| CTLA-4 IgG1 fusion protein | 125 |
| L - Histidine (200 mM) | 28 |
| L - Histidine HCl monohydrate (3 mM) | 5.6 |
| Sucrose | 110 |
| Poloxamer 188 | 8 |

| | Results | | |
|---|---|---|---|
| | 0 | 14 | 28 |
| Analytical tests | Day | Day | Day |
| % High molecular weight (HMW) specie by SEC | 0.18 | 0.93 | 1.69 |
| % Low molecular weight (LMW) species by SEC | 0.35 | 1.09 | 1.88 |

Comparison with S01 (INN PFS) where drug substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.37 | 1.38 | 1.91 |
| % Low molecular weight (LMW) species by SEC | 0.38 | 4.05 | 8.95 |

F5 shows that high concentration of histidine buffer without amino acid is able to control HMW % and LMW % in comparison to formulation F1, F2 and S01.

F5 viscosity is 7.84 cPs and osmolality is 736 mOsm/kg.

### Example 6

CTLA-4 IgG1 fusion protein injectable formulation is formulated with 125 mg/mL concentration for subcutaneous administration.

Prepared the formulation (F6) with addition of 150 mM of Histidine buffer, 110 g/L of Sucrose, 50 mM of Arginine HCl and Poloxamer (8 mg/mL) at pH 7.3±0.2. The bulk solution was filtered with 0.2 μm filter and filled 1 mL of filtered solution in 1 mL glass PFS. The composition of formulation (F6) is given below:

| Compositions | mg/mL |
|---|---|
| CTLA-4 IgG1 fusion protein | 125 |
| L - Histidine (150 mM) | 21 |
| L - Histidine HCl monohydrate (3 mM) | 4.2 |
| Sucrose | 110 |
| L-Arginine (50 mM) | 10.53 |
| Poloxamer 188 | 8 |

| | Results | | |
|---|---|---|---|
| Analytical tests | 0 Day | 14 Day | 28 Day |
| % High molecular weight (HMW) species by | 0.12 | 1.13 | *N.A. |
| SEC | | | |
| % Low molecular weight (LMW) species by | 0.38 | 0.77 | 1.37 |
| CE-SDS | | | |

*N.A. data not available due to experimental error

Comparison with S01 (INN PFS) where drug substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.37 | 1.38 | 1.91 |
| % Low molecular weight (LMW) species by SEC | 0.38 | 4.05 | 8.95 |

F6 shows that high concentration of histidine 150 mM in combination with sucrose and amino acid controls effectively HMW % and LMW % in comparison to formulation F1, F2 and S01. It is also noted that LMW % is significantly high in S01 phosphate buffer while F6 shows very low LMW % and thereby improves stability.

F6 viscosity is 8.09 cPs and osmolality is 785 mOsm/kg.

### Example 7

CTLA-4 IgG1 fusion protein injectable formulation is formulated with 125 mg/mL concentration for subcutaneous administration.

Prepare the formulation (F7) with addition of 150 mM of Histidine buffer, 110 g/L of Sucrose, 50 mM of Lysine HCl and Poloxamer (8 mg/mL) at pH 7.3±0.2. The bulk solution was filtered with 0.2 μm filter and filled in 1 mL glass PFS. The composition of formulation (F7) is given below:

| Compositions | mg/mL |
|---|---|
| CTLA-4 IgG1 fusion protein | 125 |
| L - Histidine | 21 |
| L - Histidine HCl monohydrate | 4.2 |
| Sucrose | 110 |
| L-Lysine (50 mM) | 9.13 |
| Poloxamer 188 | 8 |

| | Results | | |
|---|---|---|---|
| | 0 | 14 | 28 |
| Analytical tests | Day | day | Day |
| % High molecular weight (HMW) species by SEC | 0.14 | 0.88 | 1.7 |
| % Low molecular weight (LMW) species by | 0.37 | 0.85 | 1.46 |
| CE-SDS | | | |

Comparison with S01 (INN PFS) where drug substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | | |
|---|---|---|---|
| | 0 | 14 | 28 |
| Analytical tests | Day | Day | Day |
| % High molecular weight (HMW) species by SEC | 0.37 | 1.38 | 1.91 |
| % Low molecular weight (LMW) species by SEC | 0.38 | 4.05 | 8.95 |

F7 shows that high concentration of histidine 150 mM in combination with sucrose and amino acid controls effectively HMW % and LMW % in comparison to formulation F1, F2 and S01. It is also noted that LMW % is significantly high in S01 phosphate buffer while F6 shows very low LMW % and thereby improves stability.

F7 viscosity is 8.74 cPs and osmolality is 818 mOsm/kg.

### Example 8

CTLA-4 IgG1 fusion protein injectable formulation is formulated with 125 mg/mL concentration for subcutaneous administration.

Prepared the formulation (F8) with addition of 100 mM of Histidine buffer, 110 g/L of Sucrose, 50 mM of arginine, 50 mM of lysine and Poloxamer (8 mg/mL) at pH 7.3±0.2. The bulk solution was filtered with 0.2 μm filter and filled in 1 mL glass PFS. The composition of formulation (F8) is given below:

| Compositions | mg/mL |
|---|---|
| CTLA-4 IgG1 fusion protein | 125 |
| L - Histidine | 15.05 |
| L - Histidine HCl monohydrate | 0.63 |
| Sucrose | 110 |
| Arginine HCl | 10.53 |
| L-Lysine | 9.13 |
| Poloxamer 188 | 8 |

| | Results | |
|---|---|---|
| Analytical tests | 0 Day | 7 Day |
| % High molecular weight (HMW) species by SEC | 0.13 | 0.49 |
| % Low molecular weight (LMW) species by CE-SDS | 0.27 | 0.54 |

Comparison with S01 (INN PFS) where drug substance CTLA-4 IgG1 is added in formulation excipients as follow:

| Compositions | mg/mL |
|---|---|
| Abatacept (CTLA-4 IgG1) | 125 |
| Dibasic sodium phosphate anhydrous (6 mM) | 0.838 |
| monobasic sodium phosphate monohydrate (2 mM) | 0.28 |
| Sucrose (500 mM) | 170 |
| Poloxamer 188 | 8 |

| | Results | |
|---|---|---|
| Analytical tests | 0 Day | 7 Day |
| % High molecular weight (HMW) species by SEC | 0.37 | 0.99 |
| % Low molecular weight (LMW) species by SEC | 0.38 | 4.05 |

It is evident that S01 formulation has very high HMW % and LMW % comparison to formulation (F7).

1. A pharmaceutical liquid formulation comprising:
a. high concentration of a pharmacologically active fusion protein;
b. a histidine buffer;
c. a suitable amino acid;
d. a stabilizer;
e. a surfactant; and
f. pH selected from about 6.5 to about 7.5;

2. The pharmaceutical formulation according to claim 1, wherein the concentration of the pharmacologically active fusion protein is at least 100 mg/ml.

3. The pharmaceutical formulation according to claim 2, wherein the concentration of pharmacologically active fusion protein is about 50 mg/0.4 ml, about 87.5 mg/0.7 ml, or about 125 mg/ml.

4-5. (canceled)

6. The pharmaceutical formulation according to claim 1, wherein the histidine buffer is present from about 100 mM to about 200 mM.

7. (canceled)

8. The pharmaceutical formulation according to claim 1, wherein the concentration of the suitable amino acid is selected from about 10 mM to about 200 mM; and
wherein the suitable amino acid is selected from arginine, lysine, glycine, and proline, and a suitable salt thereof.

9-11. (canceled)

12. The pharmaceutical formulation according to claim 8, wherein the suitable amino acid is lysine; and wherein the concentration of the suitable amino acid is about 10 mM to about 100 mM.

13. The pharmaceutical formulation according to claim 12, wherein the suitable amino acid is lysine; and wherein the concentration of the suitable amino acid is about 50 mM.

14. The pharmaceutical formulation according to claim 1, wherein the stabilizer selected from sugar e and cyclodextrin.

15. The pharmaceutical formulation according to claim 14, wherein the sugar concentration is lower than concentration of the pharmacologically active fusion protein.

16. The pharmaceutical formulation according to claim 14, wherein the sugar is sucrose.

17. The pharmaceutical formulation according to claim 16, wherein the ratio of the pharmacologically active fusion protein to sucrose is not more than 1:0.9, 1:0.8, 1:0.7, 1:0.6, or 1:0.5.

18. The pharmaceutical formulation according to claim 14, wherein the concentration of sucrose is from about 110 mg/mL to about 120 mg/mL.

19. (canceled)

20. The pharmaceutical formulation according to claim 1, wherein the surfactant is selected from polysorbate and poloxamer 188.

21. (canceled)

22. The pharmaceutical formulation according to claim 20, wherein the surfactant is Poloxamer 188; and wherein the concentration of the surfactant is from about 1 mg/mL to 10 mg/mL.

23. The pharmaceutical formulation according to claim 22, wherein the surfactant is Poloxamer 188; and wherein the concentration of the surfactant is from about 5 mg/mL to 8 mg/mL.

24. The pharmaceutical formulation according to claim 1, wherein the pH of the pharmaceutical liquid formulation is about pH 6.8, about pH 6.9, about pH 7.0, about pH 7.1, about pH 7.2, about pH 7.3, or about pH 7.4.

25. (canceled)

26. The pharmaceutical formulation according to claim 1, wherein the pharmacologically active fusion protein comprises a receptor selected from CTLA4, TNFR, VEGF, HER-2, and PCSK9 fused with constant region of IgG1.

27. The pharmaceutical formulation according to claim 1, wherein the pharmacologically active fusion protein is CTLA4-Ig fusion protein.

28. (canceled)

29. The stable pharmaceutical liquid formulation according to claim 1, wherein the pharmaceutical liquid formulation comprises:
a. high concentration of the pharmacologically active fusion protein;
b. the histidine buffer;
c. lysine;
d. sucrose;
e. poloxamer 188; and
f. pH of about pH 6.8, about pH 6.9, about pH 7.0, about pH 7.1, about pH 7.2, about pH 7.3, or about pH 7.4.

30. (canceled)

31. A stable pharmaceutical liquid formulation of fusion protein comprising:
a. high concentration of pharmacologically active fusion protein;
b. histidine buffer;
c. optionally suitable amino acid;
d. sucrose;
e. poloxamer 188 and;
f. pH of about pH 6.8, about pH 6.9, about pH 7.0, about pH 7.1, about pH 7.2, about pH 7.3, or about pH 7.4.

32. A drug delivery device comprising a pharmaceutical liquid formulation according to claim 1.

33. (canceled)

34. The pharmaceutical liquid formulation according to claim 1, wherein the kinematic viscosity of the pharmaceutical liquid formulation is about 7 cps, about 8 cps, about 9 cps, about 10 cps, about 11 cps, about 12 cps, about 13 cps, about 14 cps, or about 15 cps.

35. (canceled)

36. The pharmaceutical liquid formulation according to claim 1, wherein the osmolality of the pharmaceutical liquid formulation is about 700 mOsm/kg, about 710 mOsm/kg, about 720 mOsm/kg, about 730 mOsm/kg, about 740 mOsm/kg, about 750 mOsm/kg, about 760 mOsm/kg, about 770 mOsm/kg, about 780 mOsm/kg, about 790 mOsm/kg, about 800 mOsm/kg, about 810 mOsm/kg, about 820 mOsm/kg, about 830 mOsm/kg, about 840 mOsm/kg, about 850 mOsm/kg, about 860 mOsm/kg, about 870 mOsm/kg, about 890 mOsm/kg, or about 900 mOsm/kg.

37. The pharmaceutical liquid formulation according to claim 1, wherein the pharmaceutical liquid formulation comprises reduced amount of low molecular weight (LMW) impurities compared to pharmacologically active fusion protein formulated with phosphate buffer.

38. The pharmaceutical liquid formulation according to claim 1, wherein the pharmaceutical liquid formulation is essentially free of histidine amino acid.

39. The pharmaceutical liquid formulation according to claim 1, wherein the pharmaceutical liquid formulation comprises reduced amount of high molecular weight (HMW) impurities compared to the pharmacologically active fusion protein formulated with phosphate buffer.

40. The pharmaceutical liquid formulation according to claim 1, wherein the pharmaceutical liquid formulation comprises monomer more than 95%, 96%, 97%, 98% analysed by SEC-HPLC or CE-SDS; wherein low molecular weight (LMW) impurities in an amount below 10% or 9% or 8% or 7% or 6% or 5% or 4% or 3% or 2% or 1% when stored at 25° C. for 4 weeks analysed by SEC-HPLC or CE-SDS; and wherein high molecular weight (HMW) impurities in an amount below 10% or 9% or 8% or 7% or 6% or 5% or 4% or 3% or 2% or 1% when stored at 25° C. for 4 weeks analysed by SEC-HPLC or CE-SDS.
